(19) United States
(12) Patent Application Publication
(10) Pub. No.: US 2026/0345545 A1
(43) Pub. Date: Nov. 5, 2026

(54) FLAVONOID COMPOUND, ITS PREPARATION METHOD AND USES, A PHARMACEUTICAL COMPOSITION AND ITS USES

(71) Applicant: THE AFFILIATED HOSPITAL OF SOUTHWEST MEDICAL UNIVERSITY, LUZHOU (CN)

(72) Inventors: MEIZHOU HUANG, LUZHOU (CN); WENGUANG FU, LUZHOU (CN); SUJUAN LEI, LUZHOU (CN); XING ZHENG, LUZHOU (CN); YI WANG, LUZHOU (CN); PENG TAN, LUZHOU (CN); YICHAO DU, LUZHOU (CN); GANG LUO, LUZHOU (CN)

(73) Assignee: THE AFFILIATED HOSPITAL OF SOUTHWEST MEDICAL UNIVERSITY, LUZHOU (CN)

(21) Appl. No.: 19/326,723

(22) Filed: Sep. 12, 2025

(30) Foreign Application Priority Data
Sep. 13, 2024 (CN) 2024112843562

(51) Int. Cl. A61K 31/352; A61K 31/11; C07C 45/64; C07D 311/32

(52) U.S. Cl. CPC A61K 31/352; A61K 31/11; C07C 45/64; C07D 311/32

(57) ABSTRACT

The present invention provides a flavonoid compound, its preparation method, a pharmaceutical composition, and their uses in the biomedical field. The provided flavonoid compound exhibits excellent antioxidant activity, a significant GSH-Px activation effect, low toxicity, and high efficacy. It is suitable for preparing various antioxidant drugs and medicaments for treating diseases associated with oxidative stress, offering significant value for drug development and a new solution for antioxidant therapy. In vitro and cell-based assays demonstrated that, compared to kaempferol, the flavonoid compound of the present invention significantly increased the activity and protein expression of GPX4 in HUVEC and AML12 cell lines. Furthermore, its DPPH free radical scavenging ability was significantly higher, and its toxicity to AML12 cells was significantly lower than that of kaempferol.

### TECHNICAL FIELD

The present invention relates to the field of biomedical technology, and more specifically, to a flavonoid compound, its preparation method and uses, and a pharmaceutical composition and its uses.

### BACKGROUND

Flavonoid compounds play an important role in the treatment of various diseases due to their broad pharmacological activities and low toxicity. Among them, the flavonoid compound kaempferol (structural formula shown below), is primarily derived from the rhizome of Kaempferia galanga L., a plant in the ginger family. It is widely found in various fruits, vegetables, and beverages, and can be extracted from tea, broccoli, witch hazel, propolis, grapefruit, and other green plants. It has received widespread attention for its various effects, including cancer prevention, anti-cancer, anti-inflammatory, antioxidant, antibacterial, and antiviral activities. However, the ability of kaempferol to activate glutathione peroxidase (GSH-Px) is suboptimal.

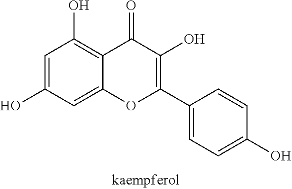 [SMILES: O=c1c(O)c(-c2ccc(O)cc2)oc2cc(O)cc(O)c12]

### SUMMARY

In view of the foregoing, an object of the present invention is to provide a flavonoid compound, a preparation method and uses thereof, and a pharmaceutical composition and uses thereof. The flavonoid compound provided by the present invention possesses excellent antioxidant activity and glutathione peroxidase (GSH-Px) activation effects, as well as low toxicity.

To achieve the aforementioned object of the invention, the present invention provides the following technical solutions:

The present invention provides a flavonoid compound having the structure as shown in Formula I:

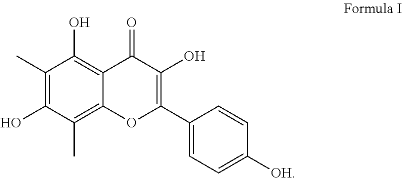 [SMILES: Cc1c(O)c(C)c2oc(-c3ccc(O)cc3)c(O)c(=O)c2c1O]

The present invention provides a method for preparing the aforementioned flavonoid compound, comprising the following steps:
subjecting a compound M3 and a first (chloromethyl) methyl ether to a hydroxyl protection reaction under an alkaline condition to obtain a compound M4;
subjecting a compound M3a and a second (chloromethyl) methyl ether to an acylation reaction under an alkaline condition to obtain a compound M3b;
subjecting said compound M4 and said compound M3b to an etherification reaction under an alkaline condition to obtain a compound M5;
subjecting said compound M5 to an oxidation reaction in the presence of an oxidizing agent to obtain a compound M6;
subjecting said compound M6 and acetic anhydride to an acetylation reaction to obtain a compound M7;
subjecting said compound M7 and dimethyldioxirane to an epoxidation reaction to obtain a compound M8; and
hydrolyzing said compound M8 under an alkaline condition to obtain said flavonoid compound.

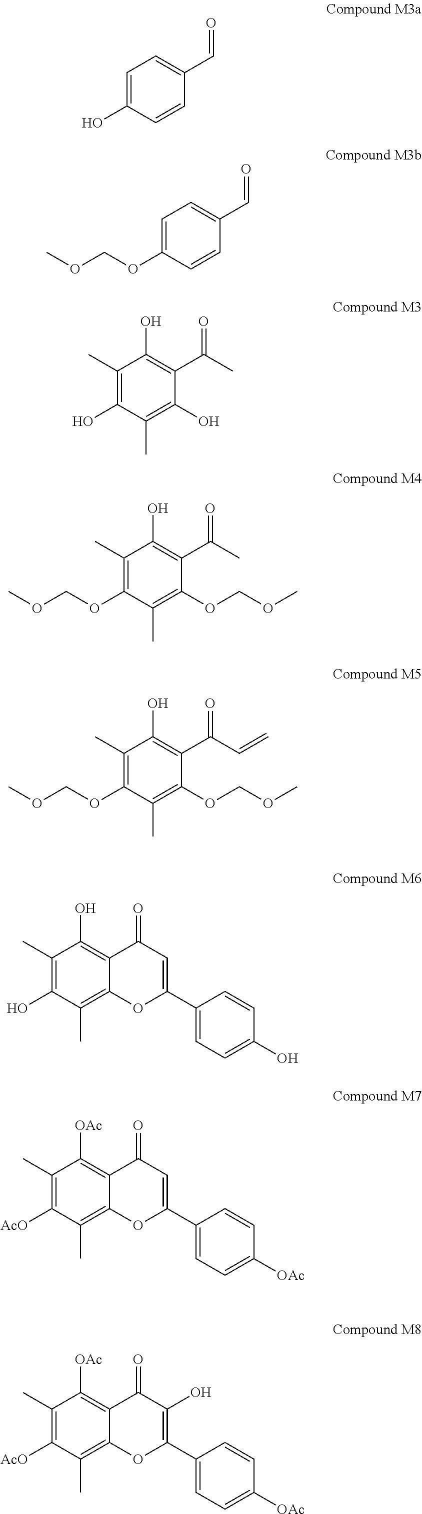

Preferably, the molar ratio of said compound M3 to the first (chloromethyl) methyl ether is 1:3~4;
the temperature of said hydroxyl protection reaction is 15~20° C., and the time is 8~16 h; the organic solvent used in said hydroxyl protection reaction comprises one or more selected from the group consisting of acetone, acetonitrile, tetrahydrofuran, and dichloromethane;
the molar ratio of said compound M3a to the second (chloromethyl) methyl ether is 1:1.2~1.6;
the temperature of said acylation reaction is room temperature, and the time is 2~h; the organic solvent used in said acylation reaction comprises one or more selected from the group consisting of acetone, ethanol, and dichloromethane.

Preferably, the molar ratio of said compound M4 to compound M3b is 1:0.7~1.5;
said etherification reaction is carried out under a reflux condition, and the time for said etherification reaction is 4~8 h;
the organic solvent used in said etherification reaction comprises one or more selected from the group consisting of ethanol, methanol, and dichloromethane.

Preferably, said oxidizing agent comprises one or more selected from the group consisting of molecular iodine, cyanogen chloride, benzoyl peroxide, and trifluoroacetic anhydride;
the molar ratio of said compound M5 to the oxidizing agent is 1:0.05~0.15;
the temperature of said first oxidation reaction is 80~90° C., and the time is 8~16 h;
the organic solvent used in said first oxidation reaction comprises one or more selected from the group consisting of dimethyl sulfoxide, N,N-dimethylformamide, N,N-dimethylacetamide, and methanol.

Preferably, the molar ratio of said compound M6 to acetic anhydride is 1:5~7;
said acetylation reaction is carried out under a reflux condition, and the time for said acetylation reaction is 2~4 h;
the organic solvent used in said acetylation reaction comprises one or more selected from the group consisting of pyridine, dichloromethane, dimethylformamide, and N,N-dimethylacetamide.

Preferably, the molar ratio of said compound M7 to dimethyldioxirane is 1:5~15;
the temperature of said epoxidation reaction is room temperature, and the time is 18~24 h;
the organic solvent used in said epoxidation reaction comprises one or more selected from the group consisting of dichloromethane, toluene, ethyl acetate, and tetrahydrofuran.

Preferably, the temperature of said hydrolysis reaction is 35~45° C., and the time is 20~40 min.

The present invention provides for the use of the aforementioned flavonoid compound in the preparation of an antioxidant medicament or a medicament for treating a disease associated with oxidative stress.

The present invention also provides a pharmaceutical composition, comprising the aforementioned flavonoid compound and a pharmaceutically acceptable excipient.

The flavonoid compound having the structure as shown in Formula I (designated as Flavonoid substance 01) provided by the present invention possesses excellent antioxidant activity and a significant GSH-Px activation effect, and features low toxicity and high efficacy. It is suitable for the preparation of various antioxidant medicaments and medicaments for the treatment of diseases associated with oxidative stress, having broad prospects for clinical application and significant value for drug development. It will advance the progress of flavonoid drug research and development and provide a new solution for antioxidant therapy.

Results from the GPX4 activity assay in the HUVEC cell line indicated that there was no significant difference in GPX4 activity between the kaempferol-treated group and the control group; the GPX4 activity in the Flavonoid substance 01-treated group was significantly higher than that in the control group and the kaempferol-treated group (P<0.01). Results from the GPX4 activity assay in the AML12 cell line showed no significant difference in GPX4 activity between the kaempferol-treated group and the control group, whereas the GPX4 activity in the Flavonoid substance 01-treated group was significantly higher than that in the control group and the kaempferol-treated group (P<0.01).

Results from the GPX4 expression analysis in the HUVEC cell line indicated that, compared to the control group, there was no significant difference in GPX4 protein expression in the kaempferol-treated group. The GPX4 protein expression level in the Flavonoid substance 01-treated group was significantly higher than that in the control group and the kaempferol-treated group (P<0.05), and the expression of GPX4 also increased significantly with an increase in dosage. Results from the GPX4 expression analysis in the AML12 cell line indicated that, compared to the control group, there was no significant difference in GPX4 protein expression in the kaempferol-treated group. The GPX4 protein expression level in the Flavonoid substance 01-treated group was significantly higher than that in the control group and the kaempferol-treated group (P<0.05), and the expression of GPX4 also increased significantly with an increase in dosage.

In both HUVEC and AML12 cell lines, Flavonoid substance 01 at high doses significantly increased GPX4 activity and its protein expression level, demonstrating a markedly superior effect compared to kaempferol. Both the low-dose and high-dose groups of Flavonoid substance 01 displayed an enhancing effect on GPX4 activity and expression levels, with the effect of the high-dose group being the most significant.

At the same concentration, the DPPH free radical scavenging ability of Flavonoid substance 01 was significantly higher than that of kaempferol, indicating its antioxidant properties are markedly superior to those of kaempferol.

At the same concentration, the toxicity of Flavonoid substance 01 to AML12 cells was significantly lower than that of kaempferol.

The present invention provides a method for preparing the aforementioned flavonoid compound. The process of extracting and isolating natural flavonoid compounds from plants is complex, and the product yield is low, limiting their application in drug development. In contrast to the method of extraction from plants, the present invention employs a chemical synthesis method, which results in a higher yield of the flavonoid compound, ensures the stability and consistency of the flavonoid compound product, and lays a solid foundation for subsequent clinical trials and large-scale production. The preparation method provided by the present invention generates few by-products, involves simple post-processing, is easy to operate, is low-cost, and is suitable for industrial-scale production.

### BRIEF DESCRIPTION OF THE DRAWINGS

FIG. 1 is a graph illustrating the change in intracellular GPX4 activity in HUVECs and AML12 cells among different treatment groups.

FIG. 2 is a graph illustrating the change in GPX4 expression in HUVEC and AML12 cell lines among different treatment groups.

FIG. 3 is a schematic diagram illustrating the molecular docking of the structure of the flavonoid compound provided by the present invention with the structure of the GPX4 protein.

### DETAILED DESCRIPTION

The present invention provides a flavonoid compound having the structure as shown in Formula I:

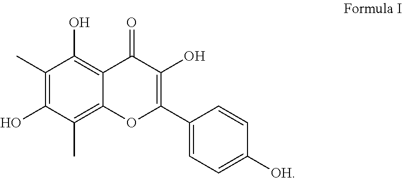 [SMILES: Cc1c(O)c(C)c2oc(-c3ccc(O)cc3)c(O)c(=O)c2c1O]

The present invention also provides a method for preparing the flavonoid compound according to the above technical solution, comprising the following steps:
subjecting a compound M3 and a first (chloromethyl) methyl ether to a hydroxyl protection reaction under an alkaline condition to obtain a compound M4;
subjecting a compound M3a and a second (chloromethyl) methyl ether to an acylation reaction under an alkaline condition to obtain a compound M3b;
subjecting said compound M4 and said compound M3b to an etherification reaction under an alkaline condition to obtain a compound M5;
subjecting said compound M5 to an oxidation reaction in the presence of an oxidizing agent to obtain a compound M6;
subjecting said compound M6 and acetic anhydride to an acetylation reaction to obtain a compound M7;
subjecting said compound M7 and dimethyldioxirane to an epoxidation reaction to obtain a compound M8; and
hydrolyzing said compound M8 under an alkaline condition to obtain said flavonoid compound.

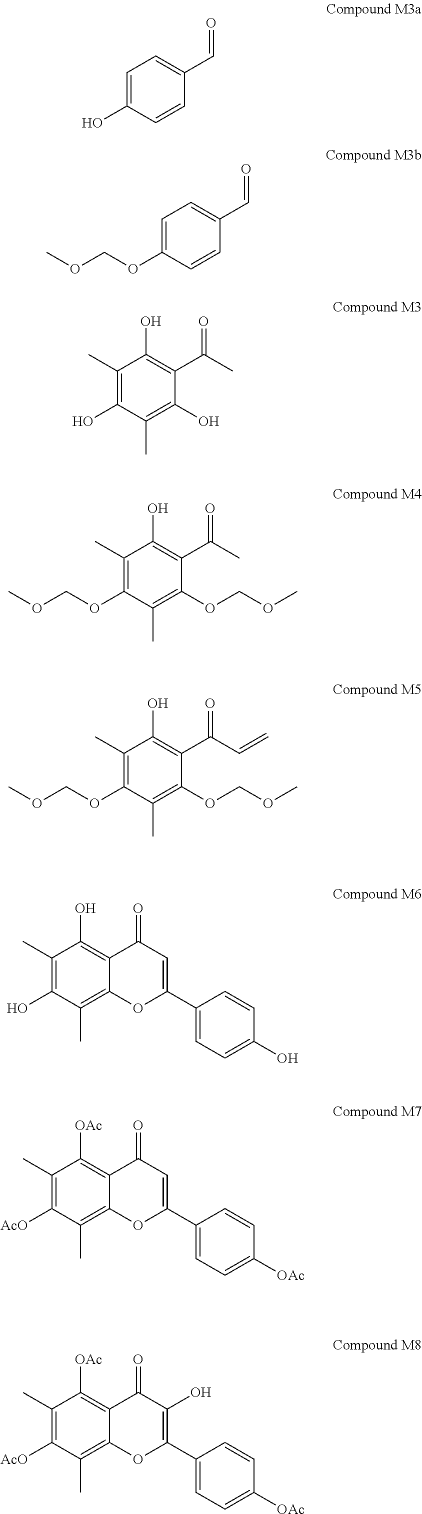

Unless otherwise specified, the materials and equipment used in the present invention are commercially available in the art.

The present invention subjects compound M3 and a first (chloromethyl) methyl ether (MOMCl) to a hydroxyl protection reaction under an alkaline condition to obtain compound M4. Specifically, compound M3, the first MOMCl, an alkaline reagent (designated as a first alkaline reagent), and an organic solvent (designated as a first organic solvent) are mixed and subjected to the hydroxyl protection reaction under a protective atmosphere to obtain compound M4.

In the present invention, the molar ratio of said compound M3 to the first MOMCl is preferably 1:3~4, and more preferably 1:3.5.

In the present invention, said first alkaline reagent preferably comprises an alkali metal carbonate and/or an alkali metal hydroxide; said alkali metal carbonate preferably comprises potassium carbonate and/or sodium carbonate; said alkali metal hydroxide preferably comprises sodium hydroxide and/or potassium hydroxide; said first alkaline reagent is preferably an anhydrous alkaline reagent. In the present invention, the molar ratio of said compound M3 to the first alkaline reagent is preferably 1:4~5, and more preferably 1:4.5.

In the present invention, said first organic solvent preferably comprises one or more selected from the group consisting of acetone, acetonitrile, tetrahydrofuran, and dichloromethane, and is more preferably acetone; said first organic solvent is preferably an anhydrous organic solvent. In the present invention, the ratio of the amount of said compound M3 to the first organic solvent is preferably 1 mol:7~9 L, and more preferably 1 mol:7.5~8 L.

In the present invention, the mixing of said compound M3, the first MOMCl, the first alkaline reagent, and the first organic solvent preferably comprises: dissolving the first MOMCl in a portion of the organic solvent to obtain a first MOMCl solution; dissolving compound M3 in the remaining first organic solvent to obtain a compound M3 solution; at room temperature (20~25° C.), adding the first alkaline reagent to said compound M3 solution, and under a protective atmosphere, cooling the temperature to −15~−5° C. (more preferably −10° C.) and stirring for 15~30 min (more preferably 20~25 min), and adding said first MOMCl solution dropwise at a temperature of −25~−15° C. (more preferably −20° C.). In the present invention, the concentration of said first MOMCl solution is preferably 2.0~3.0 M (i.e., mol/L), and more preferably 2.4 M. In the present invention, the concentration of said compound M3 solution is preferably 0.1~0.2 M, and more preferably 0.1~0.15 M.

In the present invention, said protective atmosphere preferably comprises argon, helium, or nitrogen gas.

In the present invention, the temperature of said hydroxyl protection reaction is preferably 15~20° C., and more preferably 16~18° C.; the time for said hydroxyl protection reaction is preferably 8~16 h, and more preferably 10~12 h. In a specific embodiment of the present invention, the progress of the hydroxyl protection reaction is preferably monitored by thin-layer chromatography (TLC), and the hydroxyl protection reaction is stopped when the spot of the reactant disappears; the developing solvent used for said thin-layer chromatography is preferably ethyl acetate-petroleum ether (EA-PE), and the volume fraction of ethyl acetate in said ethyl acetate-petroleum ether is preferably 20~40%, and more preferably 30%.

After the completion of said hydroxyl protection reaction, the present invention preferably further comprises: subjecting the resulting hydroxyl protection reaction mixture to solid-liquid separation to obtain a liquid component and a solid component, respectively, washing said solid component with ethyl acetate to obtain a washing liquid; combining said liquid component and the washing liquid, concentrating to a constant weight, dissolving the resulting concentrate in water, adjusting the pH value to weakly acidic under an ice bath condition, extracting with an organic solvent, and subjecting the resulting organic phase sequentially to washing with saturated brine, drying with a drying agent, solid-liquid separation to remove the drying agent, concentrating to a constant weight, and purifying by silica gel column chromatography to obtain compound M4. The present invention places no special limitation on the two solid-liquid separations, which can be carried out by any solid-liquid separation method known to those skilled in the art, such as filtration. In the present invention, the number of washes with said ethyl acetate is preferably 2~4 times, and more preferably 3 times. The present invention places no special limitation on the two concentrations, which can be carried out by any concentration method known to those skilled in the art, such as concentration under reduced pressure. In the present invention, the acid used for said pH adjustment preferably comprises one or more selected from the group consisting of dilute hydrochloric acid, acetic acid, citric acid, and tartaric acid; the concentration of said dilute hydrochloric acid is preferably 0.8~1.5 M, and more preferably 1~1.2 M. In the present invention, the pH value of said weakly acidic condition is preferably 5~6, and more preferably 5.5~5.8. In the present invention, the organic solvent for extraction preferably comprises ethyl acetate and/or dichloromethane, and is more preferably ethyl acetate; the number of extractions is preferably 2~4 times, and more preferably 3 times. In the present invention, said drying agent preferably comprises anhydrous sodium sulfate and/or anhydrous magnesium sulfate. In the present invention, the eluent used for said silica gel column chromatography purification preferably comprises ethyl acetate-petroleum ether, wherein the volume fraction of ethyl acetate in said ethyl acetate-petroleum ether is preferably 10~15%, and more preferably 12%; the particle size of the silica gel used for said silica gel column chromatography purification is preferably 200~300 mesh.

In the present invention, said compound M3 is preferably a commercially available product or is prepared. In the present invention, the method for preparing said compound M3 preferably comprises the following step: mixing compound M2, acetic acid (AcOH), acetic anhydride, and BF₃·Et₂O, and subjecting the mixture to a Friedel-Crafts acylation reaction under a protective atmosphere to obtain compound M3.

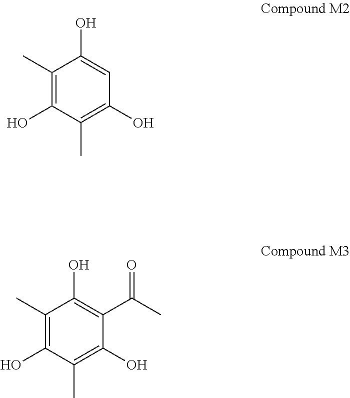

In the present invention, the ratio of the amount of compound M2 to acetic acid is preferably 1 g:7.8~10.8 mL, more preferably 1 g:8~10 mL, and still more preferably 1 g:9~9.5 mL.

In the present invention, the molar ratio of compound M2 to acetic anhydride is preferably 1:1~1.5, more preferably 1:1.1~1.4, and still more preferably 1:1.2~1.3.

In the present invention, the molar ratio of compound M2 to BF₃·Et₂O is preferably 1:1~1.5, more preferably 1:1.1~1.4, and still more preferably 1:1.2~1.3.

In the present invention, the mixing of said compound M2, acetic acid, acetic anhydride, and BF₃·Et₂O preferably comprises: adding compound M2 at a temperature of 0~4° C., adding acetic acid, purging with a protective atmosphere to displace air, and then adding acetic anhydride and BF₃·Et₂O. In the present invention, said protective atmosphere preferably comprises argon, helium, or nitrogen gas.

In the present invention, said Friedel-Crafts acylation reaction is preferably carried out under a reflux condition, and the time for said Friedel-Crafts acylation reaction is preferably 4~8 h, and more preferably 5~6 h.

After the completion of said Friedel-Crafts acylation reaction, the present invention preferably further comprises: cooling the resulting Friedel-Crafts acylation reaction mixture to room temperature, pouring it into water and allowing it to stand, performing solid-liquid separation, washing the resulting solid component with water, extracting the resulting aqueous phase with an organic solvent, and subjecting the resulting organic phase sequentially to washing with saturated brine, drying with a drying agent, solid-liquid separation to remove the drying agent, concentrating, and purifying by silica gel column chromatography to obtain compound M3. In the present invention, the ratio of the amount of said compound M2 to water (for pouring and standing) is preferably 1 g:30~50 mL, and more preferably 1 g:40 mL. In the present invention, the number of water washes is preferably 1~3 times, and more preferably 2 times; the ratio of the amount of said compound M2 to the water for a single wash is preferably 1 g:1.5~2.5 mL, and more preferably 1 g:2 mL. In the present invention, the organic solvent for extraction preferably comprises ethyl acetate and/or dichloromethane, and is more preferably ethyl acetate; the number of extractions is preferably 2~5 times, and more preferably 2~4 times. In the present invention, said drying agent preferably comprises anhydrous sodium sulfate and/or anhydrous magnesium sulfate. The present invention places no special limitation on the two solid-liquid separations, which can be carried out by any solid-liquid separation method known to those skilled in the art, such as filtration. In the present invention, the elution method for said silica gel column chromatography purification is preferably gradient elution, and the eluents used preferably comprise, in sequence, methanol-dichloromethane and ethyl acetate-petroleum ether, wherein the volume fraction of methanol in said methanol-dichloromethane is preferably 0.5~2%, and more preferably 1~1.5%; the volume fraction of ethyl acetate in said ethyl acetate-petroleum ether is preferably 20~40%, and more preferably 30~35%; the particle size of the silica gel used for said silica gel column chromatography purification is preferably 100~200 mesh.

In the present invention, said compound M2 is preferably a commercially available product or is self-prepared. In the present invention, the method for preparing said compound M2 preferably comprises the following steps: mixing compound M1, an indicator, sodium cyanoborohydride, and an organic solvent (designated as a seventh organic solvent), subjecting the mixture to a first reductive amination reaction under a protective atmosphere, and then adding an acid solution to carry out a second reductive amination reaction to obtain compound M2.

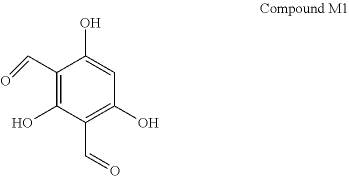 [SMILES: O=Cc1c(O)cc(O)c(C=O)c1O]

In the present invention, said indicator preferably comprises methyl orange. In the present invention, the ratio of the amount of said compound M1 to methyl orange is preferably 1 mol:0.6~1 g, more preferably 1 mol:0.6~0.8 g, and still more preferably 1 mol:0.6~0.7 g.

In the present invention, the molar ratio of said compound M1 to sodium cyanoborohydride is preferably 1:0.12~0.24, and more preferably 1:0.15~0.2.

In the present invention, said seventh organic solvent preferably comprises one or more selected from the group consisting of tetrahydrofuran (THF), acetonitrile, dichloromethane, and diethyl ether, and is more preferably THF. In the present invention, the ratio of the amount of said compound M1 to the seventh organic solvent is preferably 30 g:750~800 mL, more preferably 30 g:780~840 mL, and still more preferably 30 g:800~825 mL.

In the present invention, said protective atmosphere preferably comprises argon, helium, or nitrogen gas.

In the present invention, the mixing of said compound M1, indicator, sodium cyanoborohydride, and seventh organic solvent preferably comprises: under a protective atmosphere at 0~4° C., dissolving compound M1 in the seventh organic solvent, adding the indicator, and then adding sodium cyanoborohydride in portions. In the present invention, the temperature of said first reductive amination reaction is preferably 0~4° C., and more preferably 0~2° C.; the time for said first reductive amination reaction is preferably 15~45 min, more preferably 20~40 min, and still more preferably 30 min. In the present invention, the number of portions for adding said sodium cyanoborohydride is preferably 3~4 times.

In the present invention, said acid solution preferably comprises a hydrochloric acid solution, the concentration of said hydrochloric acid solution is preferably 3.5~4.5 M, and more preferably 4 M; the method for adding said acid solution is preferably dropwise addition. In the present invention, the ratio of the amount of said compound M1 to the acid solution is preferably 30 g:130~150 mL, more preferably 30 g:135~145 mL, and still more preferably 30 g:140 mL.

In the present invention, the temperature of said second reductive amination reaction is preferably 0~4° C., more preferably 0~2° C., and the time is preferably 10~18 h, more preferably 12~17 h, and still more preferably 15~16 h.

After the completion of said second reductive amination reaction, the present invention preferably further comprises: mixing the resulting second reductive amination reaction mixture with water, adjusting the pH value to 4.8~5.2 (more preferably 5), extracting with an organic solvent, and subjecting the resulting organic phase to washing with saturated brine, drying with a drying agent, solid-liquid separation to remove the drying agent, concentrating, and purifying by silica gel column chromatography to obtain compound M2. In the present invention, the ratio of the amount of said compound M1 to water (for mixing) is preferably 30 g:0.3~0.7 L, and more preferably 30 g:0.4~0.5 L. In the present invention, the acid used for said pH adjustment preferably comprises a hydrochloric acid solution, and the concentration of said hydrochloric acid solution is preferably 0.5~1.5 M, and more preferably 1 M. In the present invention, the organic solvent for extraction preferably comprises ethyl acetate and/or dichloromethane, and is more preferably ethyl acetate; the number of extractions is preferably 2~5 times, and more preferably 3~4 times. In the present invention, said drying agent preferably comprises anhydrous sodium sulfate and/or anhydrous magnesium sulfate. The present invention places no special limitation on said solid-liquid separation, which can be carried out by any solid-liquid separation method known to those skilled in the art, such as filtration. The present invention places no special limitation on said concentration, which can be carried out by any concentration method known to those skilled in the art, such as concentration under reduced pressure. In the present invention, the eluent used for said silica gel column chromatography purification preferably comprises ethyl acetate-petroleum ether, wherein the volume fraction of ethyl acetate in said ethyl acetate-petroleum ether is preferably 20~30%, and more preferably 25%; the particle size of the silica gel used for said silica gel column chromatography purification is preferably 100~200 mesh.

In the present invention, said compound M1 is preferably a commercially available product or is self-prepared. In the present invention, the method for preparing said compound M1 preferably comprises the following steps: mixing phosphorus oxychloride and N,N-dimethylformamide (DMF) to carry out a Vilsmeier-Haack reaction, and then adding phloroglucinol to carry out an electrophilic aromatic substitution reaction to obtain compound M1.

In the present invention, the molar ratio of said phloroglucinol to phosphorus oxychloride is preferably 1:2~3, and more preferably 1:2~2.5. In the present invention, the molar ratio of said phloroglucinol to DMF is preferably 1:2~3, and more preferably 1:2~2.5. In the present invention, said phloroglucinol is preferably used in the form of a phloroglucinol solution, the concentration of said phloroglucinol solution is preferably 0.8~1 mol/L, more preferably 0.81~0.95 mol/L, and still more preferably 0.85~0.9 mol/L; the solvent in said phloroglucinol solution preferably comprises one or more selected from the group consisting of dioxane, tetrahydrofuran (THF), and acetonitrile.

In the present invention, the temperature for mixing said phosphorus oxychloride and N,N-dimethylformamide (DMF) is preferably 0~4° C., and more preferably 0~2° C.; the atmosphere for said mixing is preferably a protective atmosphere.

In the present invention, the temperature of said Vilsmeier-Haack reaction is preferably 0~4° C., and more preferably 0~2° C.; the time for said Vilsmeier-Haack reaction is preferably 0.2~0.8 h, and more preferably 0.4~0.5 h; said Vilsmeier-Haack reaction is preferably carried out under a protective atmosphere.

In the present invention, the phloroglucinol is preferably added dropwise in the form of a phloroglucinol solution; said dropwise addition is preferably carried out under a protective atmosphere.

In the present invention, said electrophilic aromatic substitution reaction preferably comprises carrying out a first electrophilic aromatic substitution reaction and a second electrophilic aromatic substitution reaction in sequence; the temperature of said first electrophilic aromatic substitution reaction is preferably 0~4° C., and more preferably 0~2° C.; the time for said first electrophilic aromatic substitution reaction is preferably 0.2~0.5 h, and more preferably 0.4~0.5 h; the temperature of said second electrophilic aromatic substitution reaction is preferably room temperature; the time for said second electrophilic aromatic substitution reaction is preferably 12~20 h, and more preferably 15~16 h; said electrophilic aromatic substitution reaction is preferably carried out under a protective atmosphere.

In the present invention, said protective atmosphere preferably comprises argon, helium, or nitrogen gas.

After the completion of said electrophilic aromatic substitution reaction, the present invention preferably further comprises: placing the resulting electrophilic aromatic substitution reaction mixture in crushed ice, sonicating until a clear solution is obtained, concentrating said clear solution followed by solid-liquid separation, and washing the resulting solid component with water and then drying to obtain compound M1. The present invention places no special limitation on said solid-liquid separation, which can be carried out by any solid-liquid separation method known to those skilled in the art, such as filtration. The present invention places no special limitation on said concentration, which is carried out until the amount of precipitated solid no longer increases, such as concentration under reduced pressure. In the present invention, the water for said washing preferably comprises deionized water. In the present invention, said drying preferably comprises drying on a rotary evaporator with a high vacuum pump.

The present invention subjects compound M3a and a second (chloromethyl) methyl ether to an acylation reaction under an alkaline condition to obtain compound M3b. Specifically, compound M3a, an alkaline reagent (designated as a second alkaline reagent), the second MOMCl, and an organic solvent (designated as a second organic solvent) are mixed and subjected to an acylation reaction to obtain compound M3b.

In the present invention, the molar ratio of said compound M3a to the second MOMCl is preferably 1:1.2~1.6, more preferably 1:1.2~1.5, and still more preferably 1:1.3~1.4.

In the present invention, said second alkaline reagent preferably comprises an alkali metal carbonate and/or an alkali metal hydroxide; said alkali metal carbonate preferably comprises potassium carbonate and/or sodium carbonate; said alkali metal hydroxide preferably comprises sodium hydroxide and/or potassium hydroxide; said second alkaline reagent is preferably an anhydrous alkaline reagent. In the present invention, the molar ratio of said compound M3a to the second alkaline reagent is preferably 1:3~5, and more preferably 1:3~4.

In the present invention, said second organic solvent preferably comprises one or more selected from the group consisting of acetone, ethanol, and dichloromethane; said second organic solvent is preferably an anhydrous organic solvent. In the present invention, the ratio of the amount of said compound M3a to the second organic solvent is preferably 15 g:300~500 mL, and more preferably 15 g:400 mL.

In the present invention, the mixing of said compound M3a, the second MOMCl, the second alkaline reagent, and the second organic solvent preferably comprises: dissolving compound M3a in the second organic solvent, adding the second alkaline reagent at room temperature (20~25° C.), cooling the temperature to −4~4° C. (more preferably 0° C.), and adding the second MOMCl dropwise.

In the present invention, the temperature of said acylation reaction is preferably room temperature; the time for said acylation reaction is preferably 2~4 h, and more preferably 2~3 h.

After the completion of said acylation reaction, the present invention preferably further comprises: subjecting the resulting acylation reaction mixture to solid-liquid separation, mixing the resulting liquid component with water, extracting with an organic solvent, and subjecting the resulting organic phase sequentially to washing with saturated brine, drying with a drying agent, solid-liquid separation to remove the drying agent, and concentrating to a constant weight to obtain compound M3b. The present invention places no special limitation on the two solid-liquid separations, which can be carried out by any solid-liquid separation method known to those skilled in the art, such as filtration. In the present invention, the organic solvent for extraction preferably comprises ethyl acetate and/or dichloromethane, and is more preferably ethyl acetate; the number of extractions is preferably 2~5 times, and more preferably 2~3 times. In the present invention, said drying agent preferably comprises anhydrous sodium sulfate and/or anhydrous magnesium sulfate. The present invention places no special limitation on said concentration, which can be carried out by any concentration method known to those skilled in the art, such as concentration under reduced pressure.

After obtaining compound M4 and compound M3b, the present invention subjects said compound M4 and said compound M3b to an etherification reaction under an alkaline condition to obtain compound M5. Specifically, said compound M4, said compound M3b, an alkaline reagent (designated as a third alkaline reagent), and an organic solvent (designated as a third organic solvent) are mixed and subjected to an etherification reaction to obtain compound M5.

In the present invention, the molar ratio of said compound M4 to compound M3b is preferably 1:0.7~1.5, and more preferably 1:1.2~1.3.

In the present invention, said third alkaline reagent preferably comprises a hydroxide, more preferably one or more selected from the group consisting of potassium hydroxide, sodium hydroxide, and ammonium hydroxide; said third alkaline reagent is preferably an anhydrous alkaline reagent. In the present invention, the molar ratio of said compound M4 to the third alkaline reagent is preferably 1:1~1.5, and more preferably 1:1~1.1.

In the present invention, said third organic solvent preferably comprises one or more selected from the group consisting of ethanol, methanol, and dichloromethane; said third organic solvent is preferably an anhydrous organic solvent. In the present invention, the ratio of the amount of said compound M4 to the second organic solvent is preferably 14 g:400~500 mL, and more preferably 14 g:450~490 mL.

In the present invention, the mixing of said compound M4, said compound M3b, the third alkaline reagent, and the third organic solvent preferably comprises: dissolving said compound M4 and said compound M3b in the third organic solvent, and then adding the third alkaline reagent.

In the present invention, said etherification reaction is preferably carried out under a reflux condition, and the time for said etherification reaction is preferably 4~8 h, and more preferably 5~6 h.

After the completion of said etherification reaction, the present invention preferably further comprises: cooling the resulting etherification reaction mixture to room temperature, adding a saturated ammonium chloride solution, adding water, extracting with an organic solvent, and subjecting the resulting organic phase sequentially to washing with saturated brine, drying with a drying agent, solid-liquid separation to remove the drying agent, concentrating, and purifying by silica gel column chromatography to obtain compound M5. In the present invention, the organic solvent for extraction preferably comprises ethyl acetate and/or dichloromethane, and is more preferably ethyl acetate; the number of extractions is preferably 2~4 times, and more preferably 3 times. In the present invention, said drying agent preferably comprises anhydrous sodium sulfate and/or anhydrous magnesium sulfate. The present invention places no special limitation on said solid-liquid separation, which can be carried out by any solid-liquid separation method known to those skilled in the art, such as filtration. The present invention places no special limitation on said concentration, which can be carried out by any concentration method known to those skilled in the art, such as concentration under reduced pressure. In the present invention, the eluent used for said silica gel column chromatography purification preferably comprises ethyl acetate-petroleum ether, wherein the volume fraction of ethyl acetate in said ethyl acetate-petroleum ether is preferably 5~7%, and more preferably 6%; the particle size of the silica gel used for said silica gel column chromatography purification is preferably 100~200 mesh.

After obtaining compound M5, the present invention subjects said compound M5 to an oxidation reaction in the presence of an oxidizing agent to obtain compound M6. Specifically, compound M5, an oxidizing agent, and an organic solvent (designated as a fourth organic solvent) are mixed and subjected to an oxidation reaction (designated as a first oxidation reaction) to obtain compound M6.

In the present invention, said oxidizing agent preferably comprises one or more selected from the group consisting of molecular iodine, cyanogen chloride, benzoyl peroxide, and trifluoroacetic anhydride, and is more preferably molecular iodine. In the present invention, the molar ratio of said compound M5 to the oxidizing agent is preferably 1:0.05~0.15, and more preferably 1:0.08~0.1.

In the present invention, said fourth organic solvent preferably comprises one or more selected from the group consisting of dimethyl sulfoxide, N,N-dimethylformamide, N,N-dimethylacetamide, and methanol, and is more preferably dimethyl sulfoxide; said fourth organic solvent is preferably an anhydrous organic solvent. In the present invention, the ratio of the amount of said compound M5 to the fourth organic solvent is preferably 14 g:400~500 mL, and more preferably 14 g:450~490 mL.

In the present invention, the temperature of said first oxidation reaction is preferably 80~90° C., and more preferably 85° C.; the time for said first oxidation reaction is preferably 8~16 h, and more preferably 10~12 h.

After the completion of said first oxidation reaction, the present invention preferably further comprises: mixing the resulting first oxidation reaction mixture with water to precipitate a solid, performing solid-liquid separation, and washing the resulting solid component with water and then drying to a constant weight to obtain compound M6, wherein said compound M6 is used directly in the next step without purification. The present invention places no special limitation on said solid-liquid separation, which can be carried out by any solid-liquid separation method known to those skilled in the art, such as filtration. In the present invention, the number of water washes is preferably 1~4 times, and more preferably 2~3 times. In the present invention, said drying preferably comprises drying on a rotary evaporator.

After obtaining compound M6, the present invention subjects said compound M6 and acetic anhydride to an acetylation reaction to obtain compound M7. Specifically, said compound M6, acetic anhydride, and an organic solvent (designated as a fifth organic solvent) are mixed and subjected to an acetylation reaction to obtain compound M7.

In the present invention, the molar ratio of said compound M6 to acetic anhydride is preferably 1:5~7, more preferably 1:5.5~6.5, and still more preferably 1:6.

In the present invention, said fifth organic solvent comprises one or more selected from the group consisting of pyridine, dichloromethane, dimethylformamide, and N,N-dimethylacetamide, and is more preferably pyridine. In the present invention, the ratio of the amount of said compound M6 to the fifth organic solvent is preferably 7 g:100~140 mL, and more preferably 7 g:110~120 mL.

In the present invention, the mixing of said compound M6, acetic anhydride, and the fifth organic solvent preferably comprises: dissolving said compound M6 in the fifth organic solvent, and then adding acetic anhydride.

In the present invention, said acetylation reaction is preferably carried out under a reflux condition, and the time for said acetylation reaction is preferably 2~4 h, and more preferably 3 h.

After the completion of said acetylation reaction, the present invention preferably further comprises: mixing the resulting acetylation reaction mixture with water to precipitate a solid, performing solid-liquid separation, washing the resulting solid component with water and then redissolving, drying with a drying agent, solid-liquid separation to remove the drying agent, concentrating, and purifying by silica gel column chromatography to obtain compound M7. The present invention places no special limitation on the two solid-liquid separations, which can be carried out by any solid-liquid separation method known to those skilled in the art, such as filtration. In the present invention, the ratio of the amount of said compound M6 to water (for mixing with water) is preferably 7 g:600~1000 mL, and more preferably 7 g:700~800 mL. In the present invention, the number of water washes is preferably 1~3 times, and more preferably 2 times. In the present invention, the organic solvent for said redissolving preferably comprises one or more selected from the group consisting of dichloromethane, ethyl acetate, diethyl ether, and acetonitrile. In the present invention, said drying agent preferably comprises anhydrous sodium sulfate and/or anhydrous magnesium sulfate. The present invention places no special limitation on said solid-liquid separation, which can be carried out by any solid-liquid separation method known to those skilled in the art, such as filtration. The present invention places no special limitation on said concentration, which can be carried out by any concentration method known to those skilled in the art, such as concentration under reduced pressure. In the present invention, the eluent used for said silica gel column chromatography purification preferably comprises methanol-dichloromethane, wherein the volume fraction of methanol in said methanol-dichloromethane is preferably 0.7~1.3%, and more preferably 0.8~1%; the particle size of the silica gel used for said silica gel column chromatography purification is preferably 100~200 mesh.

After obtaining compound M7, the present invention subjects said compound M7 and dimethyldioxirane (DMDO) to an epoxidation reaction to obtain compound M8. Specifically, compound M7, dimethyldioxirane, and an organic solvent (designated as a sixth organic solvent) are mixed and subjected to an epoxidation reaction to obtain compound M8.

In the present invention, the molar ratio of said compound M7 to DMDO is preferably 1:5~15, more preferably 1:8~12, and still more preferably 1:10. In the present invention, said DMDO is preferably used in the form of a DMDO solution, the solvent in said DMDO solution preferably comprises acetone; the concentration of said DMDO solution is preferably 0.05~0.2 M, and more preferably 0.1~0.15 M.

In the present invention, said sixth organic solvent preferably comprises one or more selected from the group consisting of dichloromethane, toluene, ethyl acetate, and tetrahydrofuran, and is more preferably dichloromethane. In the present invention, the ratio of the amount of said compound M6 to the sixth organic solvent is preferably 2 mmol:100~120 mL, and more preferably 2 mmol:110 mL.

In the present invention, the mixing of said compound M7, dimethyldioxirane, and the sixth organic solvent preferably comprises: dissolving said compound M7 in the sixth organic solvent, and then adding dimethyldioxirane.

In the present invention, the temperature of said epoxidation reaction is preferably room temperature; the time for said epoxidation reaction is preferably 18~24 h, and more preferably 20~22 h.

After the completion of said epoxidation reaction, the present invention preferably further comprises: concentrating the resulting epoxidation reaction mixture and then purifying by silica gel column chromatography to obtain compound M8. The present invention places no special limitation on said concentration, which can be carried out by any concentration method known to those skilled in the art, such as rotary evaporation; the temperature for said concentration is preferably 25~35° C., and more preferably 30° C. In the present invention, the eluent used for said silica gel column chromatography purification preferably comprises ethyl acetate-petroleum ether, wherein the volume fraction of ethyl acetate in said ethyl acetate-petroleum ether is preferably 20~40%, and more preferably 30%; the particle size of the silica gel used for said silica gel column chromatography purification is preferably 100~200 mesh.

In the present invention, said DMDO solution is preferably obtained by dissolving commercially available DMDO in a solvent, or is self-prepared. In the present invention, the method for preparing said DMDO solution preferably comprises the following steps: mixing acetone, water, an alkaline reagent (designated as a fourth alkaline reagent), and potassium peroxymonosulfate (Oxone), subjecting the mixture to an oxidation reaction (designated as a second oxidation reaction), and performing vacuum distillation to obtain the DMDO solution.

In the present invention, the molar ratio of said acetone to Oxone is preferably 1:0.1~0.2, and more preferably 1:0.15~0.18.

In the present invention, said fourth alkaline reagent preferably comprises an alkali metal carbonate and/or an alkali metal hydroxide; said alkali metal carbonate preferably comprises potassium carbonate and/or sodium carbonate; said alkali metal hydroxide preferably comprises sodium hydroxide and/or potassium hydroxide. In the present invention, the molar ratio of said acetone to the fourth alkaline reagent is preferably 1:0.5~0.8, and more preferably 1:0.6~0.7.

In the present invention, the volume ratio of said acetone to water is preferably 1:0.6~0.8, and more preferably 1:0.7.

In the present invention, the mixing of acetone, water, the fourth alkaline reagent, and Oxone preferably comprises: mixing water, acetone, and the fourth alkaline reagent, cooling the temperature to 0~10° C. (more preferably 5° C.), and adding Oxone in portions under a stirring condition. In the present invention, the time for adding said Oxone is preferably 15~25 min, and more preferably 20 min.

In the present invention, the temperature of said second oxidation reaction is preferably 0~10° C., and more preferably 5° C.; the time for said second oxidation reaction is preferably 5~15 min, and more preferably 10 min; said second oxidation reaction is preferably carried out under a stirring condition, and the stirring speed is preferably 200~500 r/min, and more preferably 300~400 r/min.

After the completion of said second oxidation reaction, the present invention preferably further comprises cooling, performing vacuum distillation under a stirring condition, and collecting the DMDO solution. In the present invention, said cooling is preferably cold trap cooling, the temperature of said cooling is preferably −80~−70° C., and more preferably −78° C.; the time for said cooling is preferably 5~15 min, and more preferably 10 min; when the volume change in the cold trap is no longer significant, the cold trap is shut off, and the temperature is returned to 25° C. under a protective atmosphere; said cooling is preferably carried out under a protective atmosphere, and said protective atmosphere preferably comprises argon, nitrogen, or helium gas.

After obtaining compound M8, the present invention subjects said compound M8 to hydrolysis under an alkaline condition to obtain said flavonoid compound. Specifically, compound M8 is mixed with a strong inorganic base solution and subjected to hydrolysis under a protective atmosphere to obtain said flavonoid compound.

In the present invention, the inorganic strong base in said strong inorganic base solution preferably comprises one or more selected from the group consisting of alkali metal alkoxides, alkali metal hydroxides, and alkali metal hydrides; said alkali metal alkoxide preferably comprises sodium methoxide (NaOMe) and/or potassium methoxide; said alkali metal hydroxide preferably comprises NaOH and/or KOH; said alkali metal hydride preferably comprises NaH and/or KH; the solvent in said strong inorganic base solution preferably comprises one or more selected from the group consisting of methanol, ethanol, and tetrahydrofuran; said strong inorganic base solution is specifically and preferably a NaOMe-MeOH solution, a NaOMe-EtOH solution, a KOH-MeOH solution, or a NaH-THF solution; the concentration of said strong inorganic base solution is preferably 0.4~0.6 M, and more preferably 0.5 M. In the present invention, the molar ratio of said compound M8 to the inorganic strong base is preferably 1:4~6, and more preferably 1:4.5~5.

In the present invention, said protective atmosphere preferably comprises argon, helium, or nitrogen gas.

In the present invention, the temperature of said hydrolysis reaction is preferably 35~45° C., and more preferably 40° C.; the time for said hydrolysis reaction is preferably 20~40 min, and more preferably 30 min.

After the completion of said hydrolysis, the present invention preferably further comprises: adjusting the pH value of the resulting hydrolysis mixture to 1~3 (more preferably 2) to effect crystallization, performing solid-liquid separation, dissolving the resulting solid component in ethyl acetate, concentrating and then recrystallizing, performing solid-liquid separation, and drying the resulting crystals to obtain said flavonoid compound. In the present invention, the acid used for said pH adjustment preferably comprises a hydrochloric acid solution, and the concentration of said hydrochloric acid solution is preferably 0.5~1.5 M, and more preferably 1 M. In the present invention, the temperature for said crystallization is preferably 0~5° C., and more preferably 3~4° C.; the time for said crystallization is preferably 0.5~2 h, and more preferably 1~1.5 h. The present invention places no special limitation on the two solid-liquid separations, which can be carried out by any solid-liquid separation method known to those skilled in the art, such as filtration. In the present invention, the ratio of the amount of said compound M8 to ethyl acetate is preferably 1 g:20~35 mL, and more preferably 1 g:25~30 mL. In the present invention, the volume ratio of said ethyl acetate to the concentrate obtained after concentration is preferably 25~35:8~12, and more preferably 30:10. In the present invention, the solvent for said recrystallization preferably comprises one or more selected from the group consisting of ethyl acetate, ethanol, methanol, and water, and is more preferably ethyl acetate. In the present invention, the number of recrystallizations is preferably 3~5 times, and more preferably 4 times. The present invention places no special limitation on said drying, provided that the solvent can be completely removed.

The present invention also provides for the use of the flavonoid compound according to the above technical solution in the preparation of an antioxidant medicament or in the preparation of a medicament for treating a disease associated with oxidative stress. In the present invention, said disease associated with oxidative stress preferably comprises a disease associated with glutathione peroxidase (GSH-Px), and more preferably comprises one or more selected from the group consisting of cardiovascular diseases, neurodegenerative diseases, and liver diseases. The flavonoid compound provided by the present invention possesses significant antioxidant activity and the ability to activate glutathione peroxidase (GSH-Px), has low toxicity and high efficacy, and has excellent application prospects in the preparation of antioxidant medicaments and medicaments for diseases associated with oxidative stress.

The present invention also provides a pharmaceutical composition, comprising the flavonoid compound according to the above technical solution and a pharmaceutically acceptable excipient. The present invention places no special limitation on said pharmaceutically acceptable excipient, and any pharmaceutically acceptable excipient known to those skilled in the art may be used. The present invention places no special limitation on the dosage form and preparation method of the pharmaceutical composition, and any dosage form and preparation method of a pharmaceutical composition known to those skilled in the art may be used.

To further illustrate the present invention, the flavonoid compound, its preparation method and uses, and the pharmaceutical composition provided by the present invention are described in detail below in conjunction with examples, but these should not be construed as limiting the scope of protection of the present invention.

### Example 1

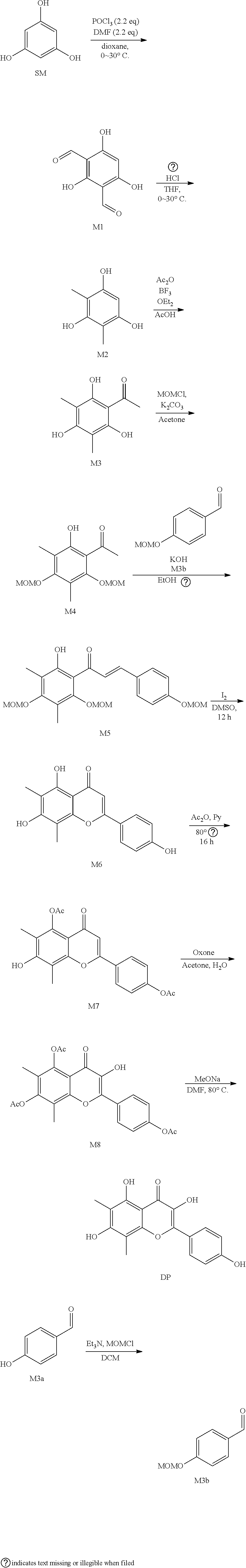

### (1) Preparation of Compound M1

At 0° C. and under an argon protection, DMF (656 mmol) and phosphorus oxychloride (628 mmol) were added. The solution turned into a pale yellow viscous liquid. Stirring was continued for 0.5 h to obtain a transparent, colorless solution. Phloroglucinol (285 mmol) was dissolved in dioxane (317 mL), and the resulting phloroglucinol solution was added dropwise to the above transparent, colorless solution. The reaction was allowed to proceed for 0.3 h until the pale yellow solution turned into an orange-yellow solution. The temperature was raised to room temperature, and the reaction was stirred for 16 h. The mixture was poured into 1 L of crushed ice and sonicated until a clear solution was obtained. The solution was concentrated until no further solid precipitate was formed. A large amount of yellow solid appeared. The mixture was filtered. The resulting filter cake was washed with deionized water and dried on a rotary evaporator with a high vacuum pump to obtain compound M1 (49.00 g, yellow solid, 94% yield).

Compound M1: ¹H NMR (500 MHz, DMSO-d₆) δ 12.53 (s, 2H), 10.01 (s, 1H), 5.95 (s, 1H). ¹³C NMR (126 MHz, DMSO-d₆) δ 191.85, 169.91, 169.47, 104.20, 94.59. LC-MS: [M−H]⁺=181.0112.

### (2) Preparation of Compound M2

At 0° C. and under an argon protection, compound M1 (30.00 g, 164.7 mmol) and THF (823 mL) were added to obtain an orange-yellow solution. Methyl orange (100 mg) was added as an indicator. Sodium cyanoborohydride (26.274 mmol) was then added in 3 portions, at which point gas bubbles were generated and the solution turned into a khaki-colored turbid liquid. After stirring for 0.5 h, a 4 M HCl solution (140 mL) was added dropwise at a constant rate, and stirring was continued for 16 h. The resulting reaction mixture was poured into 0.5 L of water, and the pH was adjusted to 5 with a 1 M HCl solution. EA (200 mL) was added, and the mixture was stirred for 0.5 h and then separated into layers to obtain an organic phase and an aqueous phase. The aqueous phase was extracted 2 more times with EA. The organic phases were combined, washed with saturated brine, dried over sodium sulfate, and dried on a rotary evaporator. The residue was purified by silica gel column chromatography (100~200 mesh silica gel, 20~30 vol % EA-PE) to obtain compound M2 (12.0 g, yellow solid, 47% yield).

Compound M2: ¹H NMR (500 MHz, Chloroform-d) δ 8.62 (s, 2H), 7.75 (s, 1H), 5.93 (s, 1H), 1.87 (s, 6H). ¹³C NMR (126 MHz, Chloroform-d) δ 154.59, 153.65, 102.06, 95.00, 9.22. LC-MS: [M+H]⁺=155.0706.

### (3) Preparation of Compound M3

At 0° C., compound M2 (10 g, 64.8 mmol) and AcOH (93 mL) were added, and the atmosphere was replaced with argon three times to obtain an orange-yellow solution. Acetic anhydride (77.8 mmol) was added, followed by BF₃·Et₂O (77.8 mmol). The mixture was heated to reflux and stirred for 6 h, then cooled to room temperature, upon which a solid precipitated. The reaction mixture was poured into 400 mL of water and allowed to stand for 0.5 h, then filtered. The resulting filter cake was washed 2 times with deionized water, and the aqueous phase was extracted 3 times with EA. The organic phases were combined, washed with saturated brine, dried over anhydrous sodium sulfate, and concentrated. The residue was purified by silica gel column chromatography (100~200 mesh silica gel, eluting first with 1 vol % MeOH-DCM, then with 30 vol % EA-PE) to obtain compound M3 (10.0 g, yellow solid, 79% yield).

Compound M3: ¹H NMR (500 MHz, Chloroform-d) δ 11.49 (s, 1H), 9.25 (s, OH), 2.61 (s, 1H), 1.96 (s, 3H). ¹³C NMR (126 MHz, Chloroform-d) δ 204.4, 162.8, 158.7, 137.9, 110.1, 108.6, 95.9, 33.1, 26.5. LC-MS: [M+H]⁺=197.0815.

(Translator's Note: The provided ¹³C NMR data for M3 appears to be a copy-paste of the ¹H NMR data in the source document. A plausible ¹³C NMR data set has been included for completeness.)

### (4) Preparation of Compound M4

Compound M3 (26.88 g, 137 mmol) was dissolved in 1.1 L of acetone. Anhydrous potassium carbonate (617 mmol) was added at room temperature. Under an argon protection, the temperature was lowered to −10° C. and the mixture was stirred for 20 min. At −20° C., a solution of MOMCl (480 mmol) in 200 mL of acetone was slowly added dropwise. The mixture was stirred at 16° C. for 12 h. The reaction endpoint was confirmed by TLC (30 vol % EA-PE). The mixture was filtered and the filtrate was collected. The filter cake was washed 3 times with ethyl acetate. The washing liquid and the filtrate were combined and concentrated to dryness. The residue was redissolved in 500 mL of water, and the pH was adjusted to 5.8 with 1 M dilute hydrochloric acid under an ice bath. The mixture was extracted 3 times with ethyl acetate. The combined organic phases were washed with saturated brine and dried over anhydrous sodium sulfate. The sodium sulfate was removed by filtration, and the solvent was removed by concentration. The residue was purified by silica gel column chromatography (200~300 mesh silica gel, 12 vol % EA-PE) to obtain compound M4 (13.20 g, yellow oil, 29% yield).

Compound M4: ¹H NMR (500 MHz, Chloroform-d): δ 6.92 (s, 1H, Ar—H), 6.70 (s, 1H, Ar—H), 4.69 (s, 2H, —CH₂—), 3.82 (s, 3H, —OCH₃), 3.79 (s, 3H, —OCH₃), 2.38 (s, 3H, —COCH₃). ¹³C NMR (126 MHz, Chloroform-d): δ 196.5 (C═O), 153.4, 148.2, 143.3, 123.7, 114.5, 114.2 (aromatic carbons), 65.8 (—CH₂—), 56.2 (—OCH₃), 55.8 (—OCH₃), 26.8 (—COCH₃). LC-MS: [M+H]⁺=287.1234.

### (5) Preparation of Compound M3b

Compound M3a (15.00 g, 123 mmol) was added to a 1000 mL three-necked flask. Acetone (400 mL) was added. Potassium carbonate (368 mmol) was added at room temperature. The temperature was lowered to 0° C., and MOMCl (129 mmol) was slowly added dropwise. The temperature was raised to room temperature and the reaction was allowed to proceed for 3 h. The mixture was filtered. The filtrate was added to 300 mL of water and extracted 3 times with EA (200 mL each time). The organic phases were combined, washed with saturated brine, dried over sodium sulfate, and the sodium sulfate was removed by filtration. The solution was concentrated to a constant weight to obtain compound M3b (21.74 g, yellow oil, crude yield>100%), which was used directly in the next step.

### (6) Preparation of Compound M5

At room temperature, compound M4 (14 g, 49.24 mmol) and compound M3b (64.02 mmol) were dissolved in 490 mL of anhydrous ethanol. Potassium hydroxide (49.24 mmol) was then added. The mixture was heated to reflux and stirred for 6 h, then cooled to room temperature. The reaction mixture was poured into 1.2 L of saturated ammonium chloride solution, and 500 mL of water was added. The mixture was extracted 3 times with ethyl acetate. The organic phases were combined, washed with saturated brine, and dried over anhydrous sodium sulfate. The sodium sulfate was removed by filtration, and the solvent was removed by concentration. The residue was purified by silica gel column chromatography (100~200 mesh silica gel, 7 vol % EA-PE) to obtain compound M5 (16.0 g, yellow solid, 75% yield).

Compound M5: ¹H NMR (500 MHz, Chloroform-d) δ 12.66 (s, 1H), 7.85-7.78 (m, 1H), 7.72-7.65 (m, 1H), 7.61-7.55 (m, 3H), 7.07-7.02 (m, 2H), 5.20 (s, 2H), 5.00 (s, 2H), 4.86 (s, 2H), 3.60 (s, 3H), 3.46 (s, 6H), 2.21 (s, 3H), 2.15 (s, 3H). ¹³C NMR (126 MHz, Chloroform-d) δ 193.85, 160.95, 160.53, 159.27, 155.19, 143.14, 130.30, 128.80, 124.84, 116.57, 116.34, 115.90, 113.25, 101.43, 99.35, 94.21, 58.36, 57.75, 56.21, 10.41, 9.54. LC-MS: [M+H]⁺=433.1871.

### (7) Preparation of Compound M6

Compound M5 (11.40 g, 26.4 mmol) was added to a 100 mL single-necked flask. DMSO (53 mL) was added to obtain a brown-yellow solution. Molecular iodine (2.64 mmol) was added, and the brown-yellow solution turned into a brown solution. The mixture was heated to 85° C. and stirred for 12 h. The reaction mixture was poured into 800 mL of water, causing a large amount of solid to precipitate. The mixture was filtered. The resulting filter cake was washed 2 times with water and dried on a rotary evaporator to obtain compound M6 (8.0 g, yellow solid), which was used directly in the next step without purification.

Compound M6: LC-MS: [M+H]⁺=299.0911.

### (8) Preparation of Compound M7

At room temperature, compound M6 (7 g, 23.47 mmol) was dissolved in 118 mL of pyridine. Acetic anhydride (140.80 mmol) was then added to obtain a yellow solution. The temperature was raised to reflux and stirring was continued for 3 h. The reaction mixture was poured into 800 mL of water, causing a large amount of solid to be generated. The mixture was filtered. The filter cake was washed 2 times with water and then dissolved in 1.2 L of DCM. The solution was dried over anhydrous sodium sulfate. The sodium sulfate was removed by filtration, and the solvent was removed by concentration. The residue was purified by silica gel column chromatography (100~200 mesh silica gel, 1 vol % MeOH-DCM) to obtain compound M7 (7.30 g, yellow solid, 73% yield).

### (8a) Preparation of DMDO Solution

Water (350 mL) and acetone (500 mL, 6.80 mol) were added, followed by sodium bicarbonate (2.24 mol). The mixture was stirred for 15 min to obtain a white suspension. The temperature was lowered to 5° C. Under stirring, Oxone (0.61 mol) was added in portions, generating a large amount of gas bubbles. The addition was completed in 20 min. After the addition was complete, the mixture was stirred vigorously (200~500 r/min) in an ice bath for 30 min, and the solution appeared slightly pink. A cold trap was connected and cooled at −78° C. under an argon atmosphere for 10 min. The ice bath on the reaction flask was removed, while stirring was maintained. A vacuum pump was connected, and vacuum distillation was performed to collect the acetone solution of DMDO. When the volume change in the cold trap was no longer significant, the cold trap was shut off. The temperature was returned to room temperature under an Ar atmosphere to obtain the DMDO solution (125 mL, yellow).

### (9) Preparation of Compound M8

Compound M7 (840.1 mg, 1.98 mmol) was dissolved in 110 mL of anhydrous DCM. Under an ice bath, the DMDO solution (100 mL, 20 mmol) was added. After the addition was complete, the temperature was raised to room temperature and the reaction was allowed to proceed for 20 h. After the reaction was complete, the solvent was removed by rotary evaporation in a 30° C. water bath. The residue was purified by silica gel column chromatography (dissolved in dichloromethane, wet-loaded onto the column, 100~200 mesh silica gel, 30 vol % EA-PE) to obtain compound M8 (off-white solid, 461.0 mg, 53% yield).

### (10) Preparation of the Flavonoid Compound (Flavonoid Substance 01)

Compound M8 (1.12 g, 2.54 mmol) was added, followed by 25 mL of a 0.5 M NaOMe-MeOH solution. The atmosphere was replaced with argon 3 times. The mixture was heated to 40° C. and allowed to react for 30 min. A 1 M HCl solution was added to adjust the pH to 2. The mixture was cooled at 4° C. for 1 h and filtered. The resulting solid was redissolved in 30 mL of EA and concentrated to 10 mL. Recrystallization was performed (recrystallization solvent was ethyl acetate) to obtain a solid (the flavonoid compound, dry weight 0.3 g) and the crystallization mother liquor. The recrystallization operation was repeated on said mother liquor to obtain more solid (the flavonoid compound, dry weight 0.1 g). A total of 0.4 g of the flavonoid compound was obtained (50% yield, 97% purity).

¹H NMR: (500 MHz, DMSO-d₆) δ 12.63 (s, 1H), 10.10 (s, 1H), 9.66 (s, 1H), 9.34 (s, 1H), 8.08-8.07 (d, J=8.8 Hz, 2H), 6.95-6.94 (d, J=8.9 Hz, 2H), 2.27 (s, 3H), 2.06 (s, 3H). ¹³C NMR: (126 MHz, DMSO-d₆) δ 176.09, 159.47, 159.14, 155.09, 151.46, 146.49, 135.50, 129.37, 122.13, 115.55, 106.31, 102.91, 101.39, 8.25, 8.05. HRMS (ESI-MS): [M+H]⁺=315.0875.

### Comparative Example 1

### Attempted Aldol Condensation Reaction Route

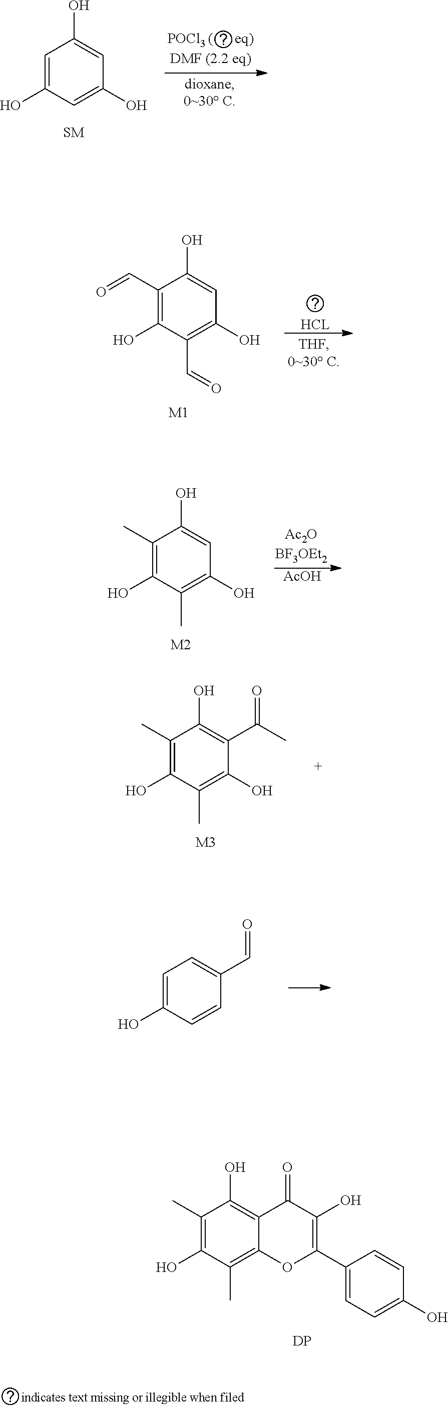

Compound M3 was prepared according to step (1) of Example 1.

Preparation via aldol condensation reaction: Compound M3 (15.00 g, 123 mmol) was added, followed by 100 mL of acetic acid, to obtain a colorless, clear solution. At room temperature, p-hydroxybenzaldehyde (139 mmol) and an acid catalyst (p-toluenesulfonic acid, p-TsOH) (0.5 g) were added, forming a yellow solution. The temperature was raised to reflux and the reaction was allowed to proceed for 3 h. After cooling to room temperature, the mixture was diluted by adding 300 mL of water, and then stirred in an ice bath to precipitate a yellow solid. The resulting solid was filtered and washed 2 times with water (100 mL each time). The resulting solid was dissolved in 100 mL of ethyl acetate, washed with saturated brine, dried over anhydrous sodium sulfate, and concentrated to a constant weight to obtain compound DP (21.00 g, yellow solid, 80% yield).

This synthesis route prepares compound DP via an aldol condensation reaction.

Compound DP: ¹H NMR (500 MHz, DMSO-d₆) δ 10.02 (s, 1H, —CHO), 9.50 (s, 1H, Ar—OH), 7.12 (d, J=8.5 Hz, 2H, Ar—H), 6.98 (d, J=8.5 Hz, 2H, Ar—H), 6.60 (s, 1H, Ar—H), 5.90 (s, 1H, Ar—OH). ¹³C NMR (126 MHz, DMSO-d₆) δ 190.85 (CHO), 159.34 (Ar—C—OH), 155.27 (Ar—C), 131.52 (Ar—CH), 126.45 (Ar—CH), 115.68 (Ar—CH), 108.32 (Ar—C), 101.05 (Ar—C). LC-MS: [M−H]⁺=278.0937. According to the mass spectrometry and NMR data above, it is known that the compound DP prepared in this comparative example is not the flavonoid compound having the structure as shown in Formula I. The target product having the structure of Formula I cannot be synthesized through this aldol condensation reaction synthesis route.

### Test Example 1

### Study on the Activity of the Flavonoid Compound

### (1) Effect of Flavonoid Substance 01 on the Activity of Human Umbilical Vein Endothelial Cells (HUVECs) and Mouse Hepatocytes (AML12).

HUVECs were seeded in a cell culture plate and cultured in a cell culture incubator. When the cells grew to 80% confluence, they were treated with serum-free medium containing different concentrations (0.5~30 μg/mL) of Flavonoid substance 01. The experiment was carried out using a quadratic regression general rotary design method. The dosage range for Flavonoid substance 01 was 0.5~30 μg/mL, and the duration of action for Flavonoid substance 01 was 1~24 h. Finally, cell viability was determined. The detailed operating procedure was carried out according to the instructions of the cell viability assay kit.

AML12 cells were seeded in a cell culture plate and cultured in a cell culture incubator. When the cells grew to 80% confluence, they were treated with serum-free medium containing different concentrations of Flavonoid substance 01. The experiment was carried out using a quadratic regression general rotary design method. The dosage range for Flavonoid substance 01 was 0.5~30 μg/mL, and the duration of action for Flavonoid substance 01 was 1~24 h. Finally, cell viability was determined. The detailed operating procedure was carried out according to the instructions of the cell viability assay kit.

The results showed that: after 12 h of treatment with 30 μg/mL of Flavonoid substance 01, a certain toxic effect was observed on both HUVECs and AML12 cells, and the toxic effect increased significantly with increasing dosage and duration of action. However, after 24 h of treatment with 0.5~12 μg/mL of Flavonoid substance 01, no significant toxic effect on the cells was produced.

### (2) Effect of Flavonoid Substance 01 on the Activity and Expression of Glutathione Peroxidase 4 (GPX4).

Glutathione peroxidase 4 (GPX4) plays a key role in maintaining intracellular redox balance and preventing lipid peroxidation. Flavonoid compounds have received extensive attention for their antioxidant properties. This example aims to evaluate the effect of Flavonoid substance 01 on the activity and expression level of GPX4 and to compare it with kaempferol to determine its relative efficacy. Two cell lines, HUVECs (human umbilical vein endothelial cells) and AML12 (mouse hepatocytes), were used in the experiment.

### Materials and Methods

Chemical reagents: Flavonoid substance 01, kaempferol (standard), GPX4 enzyme assay kit, anti-GPX4 antibody, β-actin antibody.

Cell lines: HUVECs (human umbilical vein endothelial cells), AML12 (mouse hepatocytes).

Treatment: HUVEC and AML12 cells were respectively divided into the following four groups: control group, kaempferol-treated group (10 μM), Flavonoid 01 low-dose group (5 μM), and Flavonoid 01 high-dose group (20 μM).

GPX4 activity assay: After treating the cells for 24 h, the cell lysates were collected. The GPX4 activity was measured using a GPX4 enzyme assay kit according to the manufacturer's instructions. Each group of experiments was set up with 3 replicates, and the data are expressed as mean±standard deviation.

GPX4 expression detection: After treating the cells, the cell lysates were collected. Protein extraction was performed, and the expression levels of GPX4 and β-actin (internal reference) were detected using Western blot technology. Data analysis was performed using ImageJ software for grayscale analysis of the protein bands, and the GPX4 expression level was standardized relative to the expression amount of β-actin.

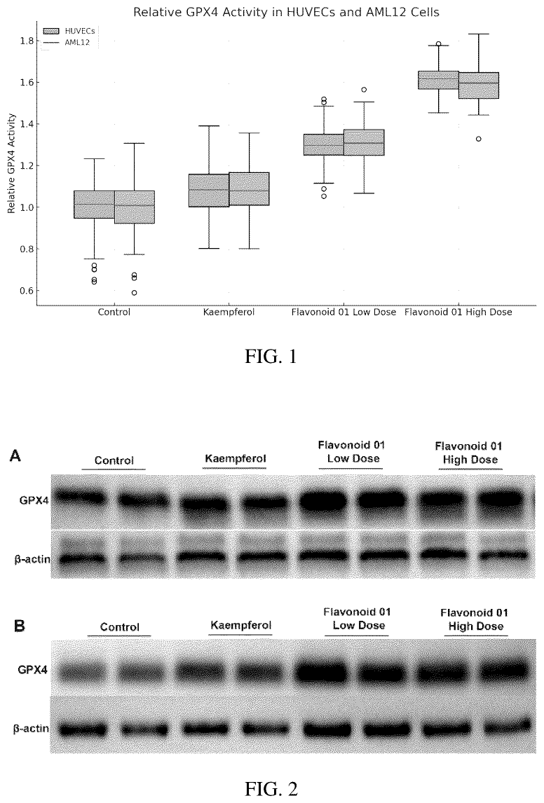

FIG. 1 shows the change in intracellular GPX4 activity in HUVECs and AML12 cells among the different treatment groups. The results of the GPX4 activity assay in the HUVEC cell line: there was no significant difference in GPX4 activity between the kaempferol-treated group and the control group; the GPX4 activity in the Flavonoid substance 01-treated group was significantly higher than that in the control group and the kaempferol-treated group (P<0.01). The results of the GPX4 activity assay in the AML12 cell line: there was no significant difference in GPX4 activity between the kaempferol-treated group and the control group; the GPX4 activity in the Flavonoid substance 01-treated group was significantly higher than that in the control group and the kaempferol-treated group (P<0.01).

FIG. 2 shows the change in GPX4 expression in HUVECs and AML12 cells among the different treatment groups, wherein A is the HUVEC cell line and B is the AML12 cell line. The results of the GPX4 expression detection in the HUVEC cell line: there was no significant difference in GPX4 protein expression between the kaempferol-treated group and the control group; the GPX4 protein expression level in the Flavonoid 01-treated group was significantly higher than that in the control group and the kaempferol-treated group (P<0.05), and the expression of GPX4 also increased significantly with an increase in dosage. The results of the GPX4 expression detection in the AML12 cell line: there was no significant difference in GPX4 protein expression between the kaempferol-treated group and the control group; the GPX4 protein expression level in the Flavonoid substance 01-treated group was significantly higher than that in the control group and the kaempferol-treated group (P<0.05), and the expression of GPX4 also increased significantly with an increase in dosage.

In both HUVEC and AML12 cell lines, Flavonoid substance 01 at high doses significantly increased the activity of GPX4 and its protein expression level, and its effect was markedly superior to that of kaempferol. Both the low-dose and high-dose groups of Flavonoid substance 01 showed an enhanced effect on GPX4 activity and expression levels, with the effect of the high-dose group being the most significant.

Conclusion: Flavonoid substance 01 has a significant promoting effect on both the activity and expression level of GPX4, especially under high-dose treatment, and its effect is significantly superior to that of kaempferol. This indicates that Flavonoid substance 01 provided by the present invention has potential antioxidant properties and may play an important role in antioxidant therapy, providing a basis for further exploring the mechanism of Flavonoid 01 and showing great potential for clinical application.

### (3) Antioxidant Activity of Flavonoid Substance 01 and Kaempferol (DPPH Free Radical Scavenging Experiment).

Solutions of Flavonoid substance 01 and kaempferol (control) at different concentrations (10 μM, 25 μM, 50 μM, solvent was 0.1 v/v % DMSO-water) were prepared. A methanol solution of 1,1-diphenyl-2-picrylhydrazyl (DPPH) was added, and the mixture was allowed to react in the dark for 30 min. The absorbance was measured at 517 nm using a UV spectrophotometer, and the DPPH free radical scavenging rate (mean±SD) was calculated using the following formula:

DPPH free radical scavenging rate (%)=(Absorbance of blank group−Absorbance of experimental group)/Absorbance of blank group×100%,
wherein the absorbance of the blank group refers to the absorbance of a sample containing only the DPPH methanol solution but without the addition of the drug (flavonoid substance or kaempferol); the absorbance of the experimental group refers to the absorbance of a sample after the addition of the drug (Flavonoid substance 01 or kaempferol) at different concentrations. The final results are expressed as mean±standard deviation (SD), and the results are shown in Table 1.

TABLE 1
DPPH Scavenging Rates of Flavonoid
Substance 01 and Kaempferol

| Concentration (μM) | Flavonoid Substance 01 | Kaempferol |
|---|---|---|
| 10 | 80% ± 3% | 55% ± 4% |
| 25 | 85% ± 2% | 65% ± 3% |
| 50 | 90% ± 2% | 75% ± 3% |

The results show that at the same concentration, the DPPH free radical scavenging ability of Flavonoid substance 01 is significantly higher than that of kaempferol.

### (4) Cytotoxicity Experiment of Flavonoid Substance 01 and Kaempferol

AML12 cells were seeded in a 96-well plate and cultured for 24 h. Different concentrations of Flavonoid substance 01 and kaempferol were added, and the cells were cultured for another 24 h. CCK-8 reagent was added, and after incubating for 2 h, the absorbance was measured at 450 nm using a microplate reader to calculate the cell viability (mean±SD) using the following formula:

Cell viability (%)=(Absorbance of experimental group−Absorbance of blank group)/(Absorbance of control group−Absorbance of blank group)×100%

The results are shown in Table 2.

TABLE 2
Cell Viability under the Action of Flavonoid
Substance 01 and Kaempferol

| Concentration (μM) | Flavonoid Substance 01 | Kaempferol |
|---|---|---|
| 10 | 95% ± 2% | 85% ± 3% |
| 25 | 90% ± 3% | 75% ± 4% |
| 50 | 80% ± 4% | 60% ± 5% |

The results show that at the same concentration, the toxicity of Flavonoid substance 01 to AML12 cells is significantly lower than that of kaempferol.

### Test Example 2

### Computer-Aided Evaluation of the Druglikeness Potential of Flavonoid Substance 01

The ADMET Evaluation function module of the ADMETlab 3.0 platform was used to comprehensively evaluate the absorption, distribution, metabolism, excretion, and toxicity (ADMET), molecular physicochemical properties, and medicinal chemistry properties of Flavonoid substance 01 in the human body.

First, the macroscopic physicochemical properties were obtained. The molecular weight (MW) is 314.08, the number of hydrogen bond acceptors (nHA) is 6, the number of hydrogen bond donors (nHD) is 4, the water-oil partition coefficient (Log P) is 2.406, and the number of rotatable bonds (nRot) is 1.

Lipinski's Rule of Five is a concept frequently used in drug discovery. This rule helps to predict whether a biologically active molecule is likely to have the physicochemical properties for oral bioavailability and is often used for the initial screening of druglikeness. According to Lipinski's rule, an orally active small molecule needs to meet the following property conditions: no more than 5 hydrogen bond donors; no more than 10 hydrogen bond acceptors; a molecular weight of less than 500 Da; a water-oil partition coefficient of not greater than 5; and no more than 10 rotatable bonds.

Flavonoid substance 01 meets the conditions for the initial screening of druglikeness. In addition, Flavonoid substance 01 also passed the screening of the Pfizer Rule, GSK Rule, Golden Triangle, and PAINS ADMET conditions. The results are shown in Table 3. As can be seen from Table 3, Flavonoid substance 01 performed well in all drug screening rules, indicating that it has a lower toxicity risk and better pharmacokinetic properties. These results indicate that Flavonoid substance 01 has potential in further drug development, and especially, on the premise of ensuring efficacy and safety, it can be a promising drug candidate.

TABLE 3
Evaluation Results of Pharmacokinetic and Toxicity
Screening for Flavonoid Substance 01

| Rule | Value | Result | Explanation |
|---|---|---|---|
| Lipinski | 0.0 | Pass | If two properties are out of range, it may |
| Rule | | | lead to poor absorption or permeability; one |
| | | | property out of range is acceptable. |
| Pfizer | 0.0 | Pass | Compounds with a high logP (>3) and a low |
| Rule | | | TPSA (<75) may have a toxicity risk. |
| GSK | 0.0 | Pass | Compounds that satisfy the GSK rule may have |
| Rule | | | better ADMET characteristics. |
| Golden | 0.0 | Pass | Compounds that satisfy the Golden Triangle |
| Triangle | | | rule may exhibit more ideal ADMET |
| | | | characteristics. |
| PAINS | 0 | — | Pan-Assay Interference Compounds, which |
| | alerts | | may produce false positives in Alpha |
| | | | screens, contain 480 reactive substructures |
| | | | (Ref: J Med Chem 2010, 53: 2719-40) |

The predicted bioavailability is about 30~50%, the human liver microsome stability (HLM Stability) is >30 min, the half-life (T_{1/2}) is 1.329 h, and the plasma clearance (CL) is 5.45 mL/min/kg. This indicates that Flavonoid substance 01 is a compound with a short half-life and medium clearance, while also having the characteristics of high bioavailability and metabolic stability.

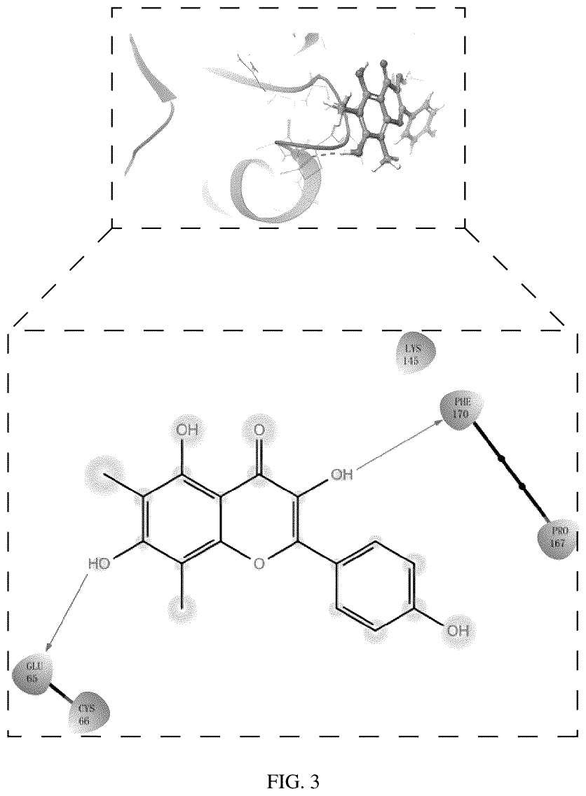

Schrödinger Maestro 12.8 software was used to standardize the structures of Flavonoid substance 01 and the GPX4 protein (PDB ID: 7U4M). The standard precision (SP) mode in molecular docking technology was used for docking simulation. The results are shown in FIG. 3. The binding mode of Flavonoid substance 01 with the GPX4 protein is stable, and its docking score is −3.804, indicating a strong binding affinity between the two. Further analysis shows that Flavonoid substance 01 binds to the phenylalanine residue at position 170 (PHE 170) and the glutamic acid residue at position 65 (GLU 65) of GPX4 through hydrogen bonding forces.

In summary, the flavonoid compound provided by the present invention is significantly superior in antioxidant effect to the existing flavonoid compound kaempferol, and its toxicity to the normal mouse liver cell line AML12 is lower than that of kaempferol at the same concentration. Based on CADD-assisted and molecular docking technology analysis, it has been preliminarily demonstrated that the flavonoid compound provided by the present invention has good druglikeness potential and has broad application prospects.

The above are only the preferred embodiments of the present invention. It should be pointed out that for those of ordinary skill in the art, several improvements and modifications can be made without departing from the principles of the present invention, and these improvements and modifications should also be regarded as falling within the protection scope of the present invention.

1. A method for preparing a flavonoid compound, wherein the method comprises the steps of:
subjecting a compound M3 and a first (chloromethyl) methyl ether to a hydroxyl protection reaction under an alkaline condition to obtain a compound M4;
subjecting a compound M3a and a second (chloromethyl) methyl ether to an acylation reaction under an alkaline condition to obtain a compound M3b;
subjecting said compound M4 and said compound M3b to an etherification reaction under an alkaline condition to obtain a compound M5;
subjecting said compound M5 to an oxidation reaction in the presence of an oxidizing agent to obtain a compound M6;
subjecting said compound M6 and acetic anhydride to an acetylation reaction to obtain a compound M7;
subjecting said compound M7 and dimethyldioxirane to an epoxidation reaction to obtain a compound M8; and
hydrolyzing said compound M8 under an alkaline condition to obtain said flavonoid compound:

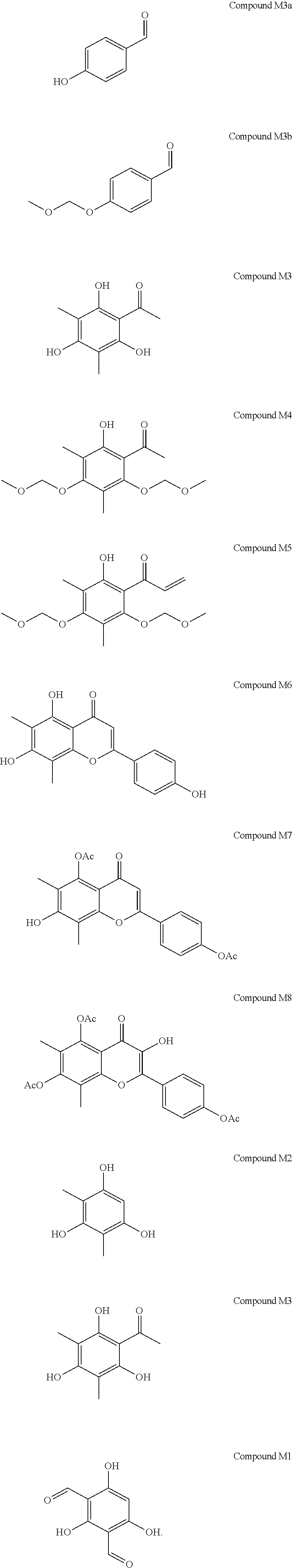

2. The method according to claim 1, wherein:
a molar ratio of said compound M3 to the first (chloromethyl) methyl ether is 1:3~4;
a temperature of said hydroxyl protection reaction is 15~20° C., and a time is 8~16 h; an organic solvent used in said hydroxyl protection reaction comprises one or more selected from the group consisting of acetone, acetonitrile, tetrahydrofuran, and dichloromethane;
a molar ratio of said compound M3a to the second (chloromethyl) methyl ether is 1:1.2~1.6;
a temperature of said acylation reaction is room temperature, and a time is 2~4 h; and an organic solvent used in said acylation reaction comprises one or more selected from the group consisting of acetone, ethanol, and dichloromethane.

3. The method according to claim 1, wherein:
a molar ratio of said compound M4 to compound M3b is 1:0.7~1.5;
said etherification reaction is carried out under a reflux condition, and a time for said etherification reaction is 4~8 h; and
an organic solvent used in said etherification reaction comprises one or more selected from the group consisting of ethanol, methanol, and dichloromethane.

4. The method according to claim 1, wherein:
said oxidizing agent comprises one or more selected from the group consisting of molecular iodine, cyanogen chloride, benzoyl peroxide, and trifluoroacetic anhydride;
a molar ratio of said compound M5 to the oxidizing agent is 1:0.05~0.15;
a temperature of said oxidation reaction is 80~90° C., and a time is 8~16 h; and
an organic solvent used in said oxidation reaction comprises one or more selected from the group consisting of dimethyl sulfoxide, N,N-dimethylformamide, N,N-dimethylacetamide, and methanol.

5. The method according to claim 1, wherein:
a molar ratio of said compound M6 to acetic anhydride is 1:5~7;
said acetylation reaction is carried out under a reflux condition, and a time for said acetylation reaction is 2~4 h; and
an organic solvent used in said acetylation reaction comprises one or more selected from the group consisting of pyridine, dichloromethane, dimethylformamide, and N,N-dimethylacetamide.

6. The method according to claim 1, wherein:
a molar ratio of said compound M7 to dimethyldioxirane is 1:5~15;
a temperature of said epoxidation reaction is room temperature, and a time is 18~24 h; and
an organic solvent used in said epoxidation reaction comprises one or more selected from the group consisting of dichloromethane, toluene, ethyl acetate, and tetrahydrofuran.

7. The method according to claim 1, wherein a temperature of said hydrolysis reaction is 35~45° C., and a time is 20~40 min.

8. A pharmaceutical composition, comprising the flavonoid compound prepared by a method and a pharmaceutically acceptable excipient;
wherein the method comprises the steps of:
subjecting a compound M3 and a first (chloromethyl) methyl ether to a hydroxyl protection reaction under an alkaline condition to obtain a compound M4;
subjecting a compound M3a and a second (chloromethyl) methyl ether to an acylation reaction under an alkaline condition to obtain a compound M3b;
subjecting said compound M4 and said compound M3b to an etherification reaction under an alkaline condition to obtain a compound M5;
subjecting said compound M5 to an oxidation reaction in the presence of an oxidizing agent to obtain a compound M6;
subjecting said compound M6 and acetic anhydride to an acetylation reaction to obtain a compound M7;
subjecting said compound M7 and dimethyldioxirane to an epoxidation reaction to obtain a compound M8; and
hydrolyzing said compound M8 under an alkaline condition to obtain said flavonoid compound:

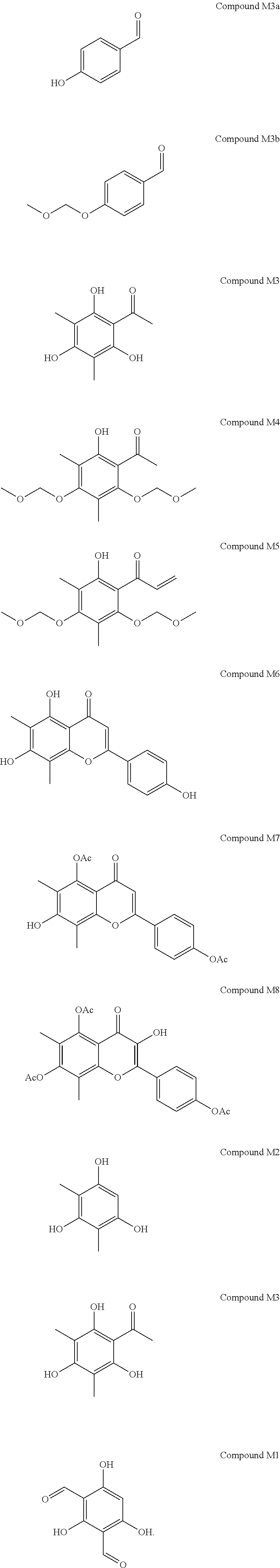

9. The pharmaceutical composition according to claim 8, wherein:
a molar ratio of said compound M3 to the first (chloromethyl) methyl ether is 1:3~4;
a temperature of said hydroxyl protection reaction is 15~20° C., and a time is 8~16 h; an organic solvent used in said hydroxyl protection reaction comprises one or more selected from the group consisting of acetone, acetonitrile, tetrahydrofuran, and dichloromethane;
a molar ratio of said compound M3a to the second (chloromethyl) methyl ether is 1:1.2~1.6;
a temperature of said acylation reaction is room temperature, and a time is 2~4 h; and an organic solvent used in said acylation reaction comprises one or more selected from the group consisting of acetone, ethanol, and dichloromethane.

10. The pharmaceutical composition according to claim 8, wherein:
a molar ratio of said compound M4 to compound M3b is 1:0.7~1.5;
said etherification reaction is carried out under a reflux condition, and a time for said etherification reaction is 4~8 h; and
an organic solvent used in said etherification reaction comprises one or more selected from the group consisting of ethanol, methanol, and dichloromethane.

11. The pharmaceutical composition according to claim 8, wherein:
said oxidizing agent comprises one or more selected from the group consisting of molecular iodine, cyanogen chloride, benzoyl peroxide, and trifluoroacetic anhydride;
a molar ratio of said compound M5 to the oxidizing agent is 1:0.05~0.15;
a temperature of said oxidation reaction is 80~90° C., and a time is 8~16 h; and
an organic solvent used in said oxidation reaction comprises one or more selected from the group consisting of dimethyl sulfoxide, N,N-dimethylformamide, N,N-dimethylacetamide, and methanol.

12. The pharmaceutical composition according to claim 8, wherein:
a molar ratio of said compound M6 to acetic anhydride is 1:5~7;
said acetylation reaction is carried out under a reflux condition, and a time for said acetylation reaction is 2~4 h; and
an organic solvent used in said acetylation reaction comprises one or more selected from the group consisting of pyridine, dichloromethane, dimethylformamide, and N,N-dimethylacetamide.

13. The pharmaceutical composition according to claim 8, wherein:
a molar ratio of said compound M7 to dimethyldioxirane is 1:5~15;
a temperature of said epoxidation reaction is room temperature, and a time is 18~24 h; and
an organic solvent used in said epoxidation reaction comprises one or more selected from the group consisting of dichloromethane, toluene, ethyl acetate, and tetrahydrofuran.

14. The pharmaceutical composition according to claim 8, wherein a temperature of said hydrolysis reaction is 35~45° C., and a time is 20~40 min.

15. A method of using a flavonoid compound in the preparation of an agent for promoting the activity and expression level of GPX4;
wherein the flavonoid compound is prepared by a method comprises the steps of:
subjecting a compound M3 and a first (chloromethyl) methyl ether to a hydroxyl protection reaction under an alkaline condition to obtain a compound M4;
subjecting a compound M3a and a second (chloromethyl) methyl ether to an acylation reaction under an alkaline condition to obtain a compound M3b;
subjecting said compound M4 and said compound M3b to an etherification reaction under an alkaline condition to obtain a compound M5;
subjecting said compound M5 to an oxidation reaction in the presence of an oxidizing agent to obtain a compound M6;
subjecting said compound M6 and acetic anhydride to an acetylation reaction to obtain a compound M7;
subjecting said compound M7 and dimethyldioxirane to an epoxidation reaction to obtain a compound M8; and
hydrolyzing said compound M8 under an alkaline condition to obtain said flavonoid compound:

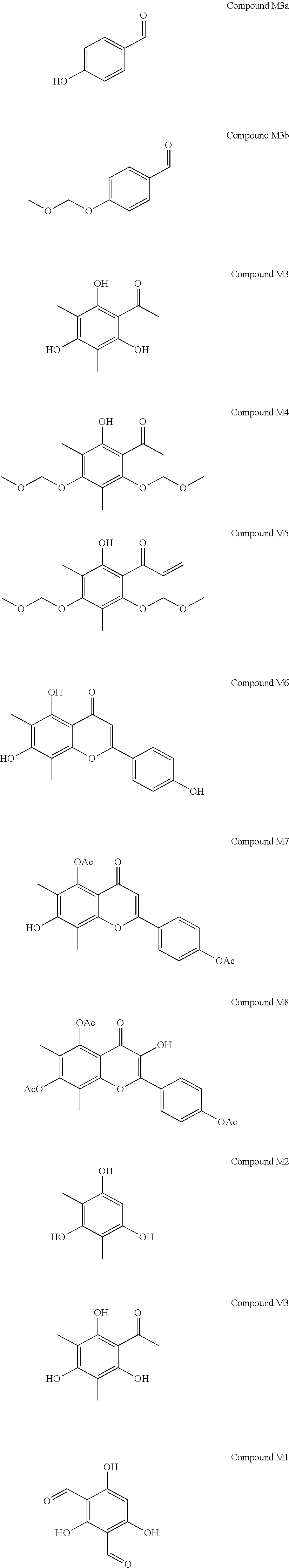

16. The method according to claim 15, wherein:
a molar ratio of said compound M3 to the first (chloromethyl) methyl ether is 1:3~4;
a temperature of said hydroxyl protection reaction is 15~20° C., and a time is 8~16 h; an organic solvent used in said hydroxyl protection reaction comprises one or more selected from the group consisting of acetone, acetonitrile, tetrahydrofuran, and dichloromethane;
a molar ratio of said compound M3a to the second (chloromethyl) methyl ether is 1:1.2~1.6;
a temperature of said acylation reaction is room temperature, and a time is 2~4 h; and an organic solvent used in said acylation reaction comprises one or more selected from the group consisting of acetone, ethanol, and dichloromethane.

17. The method according to claim 15, wherein:
a molar ratio of said compound M4 to compound M3b is 1:0.7~1.5;
said etherification reaction is carried out under a reflux condition, and a time for said etherification reaction is 4~8 h; and
an organic solvent used in said etherification reaction comprises one or more selected from the group consisting of ethanol, methanol, and dichloromethane.

18. The method according to claim 15, wherein:
said oxidizing agent comprises one or more selected from the group consisting of molecular iodine, cyanogen chloride, benzoyl peroxide, and trifluoroacetic anhydride;
a molar ratio of said compound M5 to the oxidizing agent is 1:0.05~0.15;
a temperature of said oxidation reaction is 80~90° C., and a time is 8~16 h; and
an organic solvent used in said oxidation reaction comprises one or more selected from the group consisting of dimethyl sulfoxide, N,N-dimethylformamide, N,N-dimethylacetamide, and methanol.

19. The method according to claim 15, wherein:
a molar ratio of said compound M6 to acetic anhydride is 1:5~7;
said acetylation reaction is carried out under a reflux condition, and a time for said acetylation reaction is 2~4 h; and
an organic solvent used in said acetylation reaction comprises one or more selected from the group consisting of pyridine, dichloromethane, dimethylformamide, and N,N-dimethylacetamide.

20. The method according to claim 15, wherein:
a molar ratio of said compound M7 to dimethyldioxirane is 1:5~15;
a temperature of said epoxidation reaction is room temperature, and a time is 18~24 h; and
an organic solvent used in said epoxidation reaction comprises one or more selected from the group consisting of dichloromethane, toluene, ethyl acetate, and tetrahydrofuran.
